(19) United States
(12) Patent Application Publication
(10) Pub. No.: US 2006/0217355 A1
(43) Pub. Date: Sep. 28, 2006

(54) Co-administration of dehydroepiandrosterone (DHEA) congeners and other active agents for treating cancer

(76) Inventors: Dinesh Patel, Salt Lake City, UT (US); David Vollmer, West Jordan, UT (US); Claire Daugherty, Salt Lake City, UT (US); John Gebhard, Salt Lake City, UT (US)

Correspondence Address: THORPE NORTH & WESTERN, LLP., 8180 SOUTH 700 EAST, SUITE 200, SANDY, UT, 84070, US

(21) Appl. No.: 11/370,218

(22) Filed: Mar. 6, 2006

Related U.S. Application Data
(63) Continuation-in-part of application No. 11/145,024, filed on Jun. 3, 2005.
(60) Provisional application No. 60/584,350, filed on Jun. 30, 2004.

(51) Int. Cl. A61K 31/53; A61K 31/365; A61K 31/415

(52) U.S. Cl. 514/171; 514/406; 514/471

(57) ABSTRACT

The present invention is related to therapeutic uses of dehydroepiandrosterone (DHEA) congeners. More specifically, the present invention relates to the co-administration of a dehydroepiandrosterone (DHEA) congener in combination with at least one other pharmaceutically active agent to treat or prevent cancer.

This application is a continuation-in-part of U.S. patent application Ser. No. 11/145,024, filed on Jun. 3, 2005, which claims the benefit of U.S. Provisional Application No. 60/584,350, filed on Jun. 30, 2004, both of which are incorporated herein by reference in their entireties.

### FIELD OF THE INVENTION

The present invention is drawn to methods of treating cancer. More particularly, the present invention relates to the co-administration of a dehydroepiandrosterone (DHEA) congener in combination with at least one other pharmaceutically active agent to treat cancer or reduce inflammation associated with cancer.

### BACKGROUND OF THE INVENTION

Inflammation within a human subject is a common physiological response by the immune system to an injury or irritation, where the irritation can be by infectious, allergic, and/or chemical irritants. Some of the clinically observable symptoms of inflammation include increased redness, temperature, swelling, and pain, as well as the loss of function within the inflamed area. These symptoms can be a direct result of infiltration of body fluids and leucocytes (white blood cells) into the inflamed area. This physiological response can be beneficial for the subject because of the ability of the body fluids to dilute any present toxins or substances, to facilitate the entry of antibodies, nutrients, oxygen, and immunological cells to the site, and to aid in drainage from the site. Additionally, leucocytes can aid in destroying any foreign substance within the inflamed area.

While inflammation can primarily be a favorable defense mechanism, it can also have unfavorable consequences when it is an inappropriate immunological response incited by a non-harmful substance. Cancer in particular is often associated with inappropriate inflammation. The onset of cancer in a human subject can affect natural inflammatory pathways, so as to render tissues susceptible to tumor growth and hamper immune responses that might normally hinder progression of the disease. Thus, inflammation often serves as a reliable signal in diagnosing the onset of cancer. Likewise, chronic inflammation has been identified as a risk factor for cancer.

### SUMMARY OF THE INVENTION

It has been recognized that combating inflammation generally, and inflammatory conditions associated with cancer in particular, can be an important part in treatment and prevention of cancer. The present invention provides methods of treating cancer and cancer-related inflammation that take advantage of the role of DHEA in suppressing pro-inflammatory cytokines and other pathways and mechanisms. A method of treating a subject suffering from cancer or from inflammation associated with cancer can comprise co-administering to the subject a therapeutically effective amount of a DHEA congener and a second anti-inflammatory agent. In a particular aspect of the invention, the method may be employed to treat a subject suffering from a particular cancerous disorder. Though many second anti-inflammatory agents are useful for treating various types of cancer, the second anti-inflammatory agent that can be used includes COX-2 inhibitors, such as celecoxib, rofecoxib, valdecoxib, parecoxib, lumiracoxib, and combinations thereof. According to one aspect of the invention, co-administration of DHEA and the second anti-inflammatory agent may be more effective than administration of either alone in treating cancer.

The present invention also provides methods of treating pancreatic cancer in a subject, comprising co-administering a DHEA congener and a COX-2 inhibitor to the subject. According to an embodiment of the invention, inhibiting the growth of pancreatic cancer in a subject who is determined to have a predisposition to that disease may comprise preemptively co-administering the DHEA congener and the COX-2 inhibitor to the subject before the subject is diagnosed with that disease. According to another embodiment, such preemptive co-administration reduces inflammation of the pancreas. In yet another embodiment, inhibiting growth of pancreatic cancer includes slowing the proliferation of pancreatic cancer cells. In still another aspect, associated with the inhibiting of cancer cell growth is a method where the presence of pancreatic cells is reversed.

Additional features and advantages of the invention will be apparent from the detailed description, which illustrates, by way of example, features of the invention.

### BRIEF DESCRIPTION OF THE DRAWINGS

FIG. 1 is a graphical representation depicting DHEA and sulfasalazine alone or in combination on in vivo TNF-α in a TNBS IBD animal model; and

FIG. 2 is a graphical representation depicting the myeloperoxidase levels from the same animal model.

### DETAILED DESCRIPTION OF THE INVENTION

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and “the” include plural referents unless the context clearly dictates otherwise. Thus, for example, reference to “a drug” includes reference to one or more of such drugs.

As used herein, the terms “formulation” and “composition” may be used interchangeably and refer to a combination of a pharmaceutically active agent, such as a DHEA congener, with one or more additional anti-inflammatory agent. The terms “drug,” “active agent,” “bioactive agent,” “pharmaceutically active agent,” and “pharmaceutical,” can also be used interchangeably to refer to an agent or substance that has measurable specified or selected physiologic activity when administered to a subject in an effective amount. These terms of art are well known in the pharmaceutical and medicinal arts.

As used herein, “administration” and “administering” refer to the manner in which a drug, formulation, or composition is introduced into the body of a subject. Administration can be accomplished by various art-known routes such as oral, parenteral, transdermal, inhalation, implantation, etc. Thus, an oral administration can be achieved by swallowing, chewing, or sucking of an oral dosage form comprising the drug. Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. These and additional methods of administration are well known in the art.

The term “co-administering,” co-administration,” or “co-administer” refers to the administration of a DHEA congener with another anti-inflammatory active agent. Both the DHEA congener and the second anti-inflammatory active agent can be administered simultaneously, or at different times, as long as these active agents work in concert to produce a physiological effect. Additionally, co-administration does not require the DHEA congener and the second anti-inflammatory active agent to be administered by the same route. As such, each can be administered independently or as a common dosage form.

The terms “effective amount” and “sufficient amount” may be used interchangeably and refer to an amount of an ingredient which, when included in a composition, is sufficient to achieve an intended compositional or physiological effect. Thus, a “therapeutically effective amount” refers to a non-toxic, but sufficient amount of an active agent, to achieve therapeutic results in treating a condition for which the active agent is known to be effective. Various biological factors may affect the ability of a substance to perform its intended task. Therefore, an “effective amount” or a “therapeutically effective amount” may be dependent on such biological factors. Further, while the achievement of therapeutic effects may be measured by a physician or other qualified medical personnel using evaluations known in the art, it is recognized that individual variation and response to treatments may make the achievement of therapeutic effects a subjective decision. In some instances, a “therapeutically effective amount” of a drug can achieve a therapeutic effect that is measurable by the subject receiving the drug. The determination of an effective amount is well within the ordinary skill in the art of pharmaceutical, medicinal, and health sciences. See, for example, Meiner and Tonascia, “Clinical Trials: Design, Conduct, and Analysis,” Monographs in Epidemiology and Biostatistics, Vol. 8 (1986), which is incorporated herein by reference.

As used herein, the terms “inhibit” or “inhibiting” refer to the process of holding back, suppressing or restraining so as to block, prevent, limit, or decrease a rate of action or function. The use of the term is not to be misconstrued to be only of absolute prevention, but can be a referent of from any incremental step of limiting or reducing a function to the full and absolute prevention of the function. For example, when the term “inhibit” or any derivative thereof is utilized in combination with any substance, such as an immune mediator responsive to TNF-α, inhibiting the substance can include the reduction of the production and/or the secretion of the substance.

As used herein, “reduce” or “reducing” refers to the process of decreasing, diminishing, or lessening, as in extent, amount, or degree of that which is reduced. The use of the term with respect to inflammation can include any incremental step that results in less inflammation, such as less redness, temperature, swelling, and/or pain. Additionally, the use of the term can include from any minimal decrease to absolute abolishment of a physiological process or effect.

As used herein, “treat,” “treatment,” or “treating” refers to the process or result of giving medical aid to a subject, where the medical aid can counteract a malady, a symptom thereof, or other related adverse physiological manifestation. Additionally, these terms can refer to the administration or application of remedies to a patient or for a disease or injury, such as a medicine or a therapy. Accordingly, the substance or remedy so applied, such as the process of providing procedures or applications, is intended to relieve illness, injury, or inflammation. Additionally, the term can be used for the procedure of preemptively acting to prevent the malady, a symptom thereof, or other related adverse physiological manifestation. As such, a treatment can be administered prior to the subject experiencing any symptoms so that the symptoms are not manifested in the subject.

As used herein, “carrier” or “inert carrier” refers to a polymeric carrier, or other carrier vehicle with which a bioactive agent, such as a DHEA congener and/or other anti-inflammatory agents, may be combined to achieve a specific dosage form. As a general principle, carriers do not substantially react with the bioactive agent in a manner which substantially degrades or otherwise adversely affects the bioactive agent or its therapeutic potential.

As used herein, “subject” refers to a mammal that may benefit from the administration of an inflammation reducing drug, a combination of drugs, or a formulation, or from a method for achieving reduced inflammation recited herein. Most often, the subject will be a human.

The term “dehydroepiandrosterone congener” or “DHEA congener” includes dehydroepiandrosterone (a.k.a. DHEA and (3β)-3-hydroxyandrost-5-en-17-one), derivatives of DHEA, metabolites of DHEA, metabolites of DHEA derivatives, salts of DHEA, salts of DHEA derivatives, etc. DHEA, generally, is a weak androgen that serves as the primary precursor in the biosynthesis of both androgens and estrogens. Typically, a DHEA congener used in accordance with embodiments of the present invention is in a pharmaceutically acceptable form.

As used herein, “mc” or “micro” when used in combination with a unit of measurement denotes the standard unit to be divided by one million, or multiplied by 1×10⁻⁶. Accordingly, the prefix “micro,” which is well known by one of ordinary skill in the art can be referred herein by the abbreviation “mc.”

As used herein, “mg/kg” or any other mass unit divided by another mass unit when used to describe a drug dose or dosing regimen denotes the mass of drug delivered per mass of the subject being administered the drug. Such use of units when referring to pharmaceuticals and their associated doses is well known to one of ordinary skill in the art.

As used herein, “mg/m²” or any other mass unit divided by an area unit when used to describe a drug dose or dosing regimen denotes the mass of the drug delivered per surface area of the subject being administered the drug. The use of mass of drug per surface area of subject when referring to pharmaceuticals and their associated doses is well known to one of ordinary skill in the art.

As used herein, “enhance” or “enhancing” of an anti-inflammatory response refers to the interaction of two or more active agents or drugs so that their combined physiological effect is greater than each of their respective individual effects.

The term “about” when referring to a numerical value or range is intended to encompass the values resulting from experimental error that can occur when taking measurements.

It is noted that though the present application discusses pancreatic cancer and pancreatitis in more detail than other types of cancer and inflammation, the treatment of cancer and inflammation associated with cancer is not so limited. To the extent that the co-administration of a DHEA-congener and a second anti-inflammatory agent is effective for treating other cancer types and other inflammation types associated with cancer, this combination of therapeutic agents can be used. In other words, the discussion of pancreatic cancer, pancreatitis, and other cancer pathways herein provides merely an exemplary teaching that favorably sets forth principles of the present invention.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of about 1 wt % to about 20 wt % should be interpreted to include not only the explicitly recited concentration limits of 1 wt % to about 20 wt %, but also to include individual concentrations such as 2 wt %, 3 wt %, 4 wt %, and sub-ranges such as 5 wt % to 15 wt %, 10 wt % to 20 wt %, etc.

A) Dehydroepiandrosterone Congeners

As stated, a DHEA congener includes DHEA (3β)-3-hydroxyandrost-5-en-17-one), derivatives of DHEA, metabolites of DHEA, metabolites of DHEA derivatives, salts of DHEA, salts of DHEA derivatives, etc. Typically, a DHEA congener used in accordance with embodiments of the present invention is in a pharmaceutically acceptable form. Examples of DHEA congeners include, but are not limited to, compounds having the general formula I, and their metabolites and pharmaceutically acceptable salts thereof: 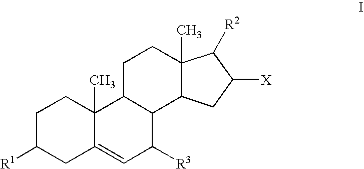
wherein

X is H or halogen;

R¹, R² and R³ are independently ═O, —OH, —SH, H, halogen, pharmaceutically acceptable ester, pharmaceutically acceptable thioester, pharmaceutically acceptable ether, pharmaceutically acceptable thioether, pharmaceutically acceptable inorganic esters, pharmaceutically acceptable monosaccharide, disaccharide or oligosaccharide, spirooxirane, spirothirane, —OSO₂R⁴ or —OPOR⁴R⁵;

R⁴ and R⁵ are independently —OH, pharmaceutically acceptable esters or pharmaceutically acceptable ethers.

Suitable metabolites of DHEA include, but are not limited to, dehydroepiandrosterone sulfate, 16α-hydroxydehydroepiandrosterone, 16α-hydroxyandrost-4-ene-3,17dione, androst-4-ene-3,17 dione, 7α-hydroxyandrostenedione, 7α-hydroxytestosterone.

Further examples of DHEA congeners, include but are not limited to, compounds having the general formulas II and III, and their metabolites and pharmaceutically acceptable salts thereof: 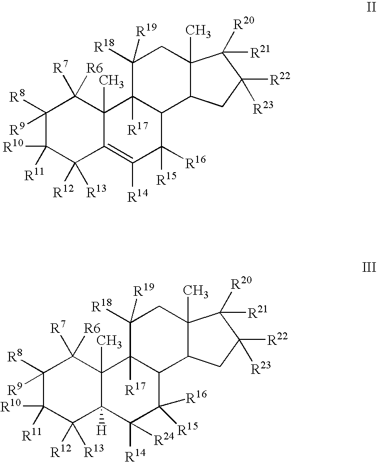
wherein
R⁶, R⁷, R⁸, R⁹, R¹¹, R¹², R¹³, R¹⁴, R¹⁵, R¹⁶, R¹⁷, R¹⁸, R¹⁹ and R²⁴ are independently H, —OH, halogen, C₁₋₁₀ alkyl or C₁₋₁₀ alkoxy;
R¹⁰ is H, —OH, halogen, C₁₋₁₀ alkyl, or C₁₋₁₀ alkoxy;
R²⁰ is (1) H, halogen, C₁₋₁₀ alkyl or C₁₋₁₀ alkoxy when R²¹ is —C(O)OR²⁵ or
(2) H, halogen, OH or C₁₋₁₀ alkyl when R²¹ is H, halogen, OH or C₁₋₁₀ alkyl or
(3) H, halogen, C₁₋₁₀ alkyl, C₁₋₁₀ alkenyl, C₁₋₁₀ alkynyl, formyl, C₁₋₁₀ alkanoyl or epoxy when R²¹ is OH; or
R²⁰ and R²¹ taken together are ═O;
R²² and R²³ are independently (1) H, —OH, halogen, C₁₋₁₀ alkyl or C₁₋₁₀ alkoxy when R²¹ is H, OH, halogen, C₁₋₁₀ alkyl or —C(O)OR²⁵ or (2) H, (C₁₋₁₀ alkyl)ₙamino, (C₁₋₁₀ alkyl)ₙamino-C₁₋₁₀ alkyl, C₁₋₁₀ alkoxy, hydroxy-C₁₋₁₀ alkyl, C₁₋₁₀ alkoxy-C₁₋₁₀ alkyl, (halogen)ₘ-C₁₋₁₀ alkyl, C₁₋₁₀ alkanoyl, formyl, C₁₋₁₀ carbalkoxy or C₁₋₁₀ alkanoyloxy when R²⁰ and R²¹ taken together are ═O; or
R²² and R²³ taken together are ═O or taken together with the carbon to which they are attached form a 3-6 member ring containing 0 or 1 oxygen atom; or
R²⁰ and R²² taken together with the carbons to which they are attached form an epoxide ring;
R²⁵ is H, (halogen)ₘ-C₁₋₁₀ alkyl or C₁₋₁₀ alkyl;
n is 0, 1 or 2;
m is 1, 2 or 3; and
physiologically acceptable salts thereof,

with the provisos that
(a) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ and R²¹ taken together are ═O; and
(b) R¹⁰ is not H, halogen, or C₁₋₁₀ alkoxy when R⁶, R⁷, R⁸, R⁹, R¹¹, R¹², R¹³, R¹⁴, R¹⁵, R¹⁷, R¹⁸, R¹⁹ and R²² are H and R¹⁶ is H, halogen, OH or C₁₋₁₀ alkoxy and R²³ is H or halogen and R²⁰ is H and R²¹ is H, OH or halogen.

The compounds represented by the general formula I exist in many stereoisomers and the formula is intended to encompass the various stereoisomers. Examples of suitable DHEA congeners of Formula I include compounds in which:

(1) R² is ═O, R³ and X are each H and R¹ is ═O, —OH, pharmaceutically acceptable esters thereof, pharmaceutically acceptable ethers thereof or pharmaceutically acceptable salts;

(2) R² is ═O, R³ is H, X is halogen and R¹ is ═O, —OH, pharmaceutically acceptable esters thereof, pharmaceutically acceptable ethers thereof or pharmaceutically acceptable salts;

(3) R² is ═O, R³ and X are each H and R¹ is —SH, pharmaceutically acceptable thioesters thereof, pharmaceutically acceptable thioethers thereof or pharmaceutically acceptable salts;

(4) R² is ═O, R³ is H, X is halogen and R¹ is —SH, pharmaceutically acceptable thioesters thereof, pharmaceutically acceptable thioethers thereof or pharmaceutically acceptable salts;

(5) R² is ═O, X is H and R¹ and R³ are independently ═O, —OH, pharmaceutically acceptable esters thereof, pharmaceutically acceptable ethers thereof or pharmaceutically acceptable salts;

(6) R² is ═O, X is halogen and R¹ and R³ are independently ═O, —OH, pharmaceutically acceptable esters thereof, pharmaceutically acceptable ethers thereof or pharmaceutically acceptable salts;

(7) R² is ═O, X is H and R¹ and R³ are independently —SH, pharmaceutically acceptable thioesters thereof, pharmaceutically acceptable thioethers thereof or pharmaceutically acceptable salts;

(8) R² is ═O, X is halogen and R¹ and R³ are independently —SH, pharmaceutically acceptable thioesters thereof, pharmaceutically acceptable thioethers thereof or pharmaceutically acceptable salts;

(9) R² is —OH, R³ and X are each H and R¹ is ═O, —OH, pharmaceutically acceptable esters thereof, pharmaceutically acceptable ethers thereof or pharmaceutically acceptable salts;

(10) R² is —OH, R³ is H, X is halogen and R¹ is ═O, —OH, pharmaceutically acceptable esters thereof, pharmaceutically acceptable ethers thereof or pharmaceutically acceptable salts;

(11) R² is —OH, R³ and X are each H and R¹ is —SH, pharmaceutically acceptable thioesters thereof, pharmaceutically acceptable thioethers thereof or pharmaceutically acceptable salts;

(12) R² is —OH, R³ is H, X is halogen and R¹ is —SH, pharmaceutically acceptable thioesters thereof, pharmaceutically acceptable thioethers thereof or pharmaceutically acceptable salts;

(13) R² is —OH, X is H and R¹ and R³ are independently ═O, —OH, pharmaceutically acceptable esters thereof, pharmaceutically acceptable ethers thereof or pharmaceutically acceptable salts;

(14) R² is —OH, X is halogen and R¹ and R³ are independently ═O, —OH, pharmaceutically acceptable esters thereof, pharmaceutically acceptable ethers thereof or pharmaceutically acceptable salts;

(15) R² is —OH, X is H and R¹ and R³ are independently —SH, pharmaceutically acceptable thioesters thereof, pharmaceutically acceptable thioethers thereof or pharmaceutically acceptable salts;

(16) R² is —OH, X is halogen and R¹ and R³ are independently —SH, pharmaceutically acceptable thioesters thereof, pharmaceutically acceptable thioethers thereof or pharmaceutically acceptable salts;

(17) R² is —SH, R³ and X are each H and R¹ is ═O, —OH, pharmaceutically acceptable esters thereof, pharmaceutically acceptable ethers thereof or pharmaceutically acceptable salts;

(18) R² is —SH, R³ is H, X is halogen and R¹ is ═O, —OH, pharmaceutically acceptable esters thereof, pharmaceutically acceptable ethers thereof or pharmaceutically acceptable salts;

(19) R² is —SH, R³ and X are each H and R¹ is —SH, pharmaceutically acceptable thioesters thereof, pharmaceutically acceptable thioethers thereof or pharmaceutically acceptable salts;

(20) R² is —SH, R³ is H, X is halogen and R¹ is —SH, pharmaceutically acceptable thioesters thereof, pharmaceutically acceptable thioethers thereof or pharmaceutically acceptable salts;

(21) R² is —SH, X is H and R¹ and R³ are independently ═O, —OH, pharmaceutically acceptable esters thereof, pharmaceutically acceptable ethers thereof or pharmaceutically acceptable salts;

(22) R² is —SH, X is halogen and R¹ and R³ are independently ═O, —OH, pharmaceutically acceptable esters thereof, pharmaceutically acceptable ethers thereof or pharmaceutically acceptable salts;

(23) R² is —SH, X is H and R¹ and R³ are independently —SH, pharmaceutically acceptable thioesters thereof, pharmaceutically acceptable thioethers thereof or pharmaceutically acceptable salts;

(24) R² is —SH, X is halogen and R¹ and R³ are independently —SH, pharmaceutically acceptable thioesters thereof, pharmaceutically acceptable thioethers thereof or pharmaceutically acceptable salts;

(25) X is H and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;

(26) X is halogen and R¹ is ═O, —OH, pharmaceutically acceptable esters thereof, pharmaceutically acceptable ethers thereof or pharmaceutically acceptable salts, R² and R³ are independently ═O, —OH, a sugar residue, pharmaceutically acceptable esters thereof, pharmaceutically acceptable ethers thereof or pharmaceutically acceptable salts, wherein at least one of R² and R³ is a sugar residue;

(27) X is H, R¹ is ═O or —OH, and R² and R³ are independently ═O, —OH, pharmaceutically acceptable inorganic esters thereof or pharmaceutically acceptable salts, wherein at least one of R² and R³ is an inorganic ester; and/or

(28) X is halogen R¹ is ═O or —OH, and R² and R³ are independently ═O, —OH, pharmaceutically acceptable inorganic esters thereof or pharmaceutically acceptable salts, wherein at least one of R² and R³ is an inorganic ester.

Pharmaceutically acceptable esters or thioesters include, but are not limited to, esters or thioesters of the formula —OOCR or —SOCR, wherein R is a pharmaceutically acceptable alkyl, alkenyl, aryl, alkylaryl, arylalkyl, sphingosine or substituted sphingolipid groups, such as propionate, enanthate, cypionate, succinate, decanoate and phenylpropionate esters.

Pharmaceutically acceptable ethers or thioethers include, but are not limited to, ethers or thioethers of the formula —OR or —SR, wherein R is as defined above or enol, or —OR is an unsubstituted or substituted spirooxirane or —SR is a spirothiane.

Suitable sugar residues can include, but are not limited to monosaccharides, disaccharides, and oligosaccharides, such as a glucuronate.

Pharmaceutically acceptable inorganic esters include, but are not limited to, esters of the formula —OSO₂R⁴ or —OPOR⁴R⁵, wherein R⁴ and R⁵ are independently —OH, pharmaceutically acceptable esters, pharmaceutically acceptable ethers or pharmaceutically acceptable salts.

Some DHEA congeners, such as the compounds of general formulas I, II, and III, can be synthesized as described in U.S. Pat. Nos. 4,898,694; 5,001,119; 5,028,631; and 5,175,154, which are all incorporated herein by reference. The compounds represented by the general formulas II and III exist in many stereoisomers and these formulas are intended to encompass the various stereoisomers. Examples of representative compounds, which fall within the scope of general formulas II and III, include the following: 5α-androstan-17-one; 16α-fluoro-5α-androstan-17-one; 3β-methyl-5α-androsten-17-one; 16β-fluoro-5α-androstan-17-one; 17β-bromo-5-androsten-16-one; 17β-fluoro-3β-methyl-5-androsten-16-one; 17α-fluoro-5α-androstan-16-one; 3β-hydroxy-5-androsten-17-one; 17α-methyl-5α-androstan-16-one; 16α-methyl-5-androsten-17-one; 3β,16α-dimethyl-5-androsten-17-one; 3β,17α-dimethyl-5-androsten-16-one; 16α-hydroxy-5-androsten-17-one; 16α-fluoro-16β-methyl-5-androsten-17-one; 16α-methyl-5α-androstan-17-one; 16-dimethylaminomethyl-5α-androstan-17-one; 16β-methoxy-5-androsten-17-one; 16α-fluoromethyl-5-androsten-17-one; 16-methylene-5-androsten-17-one; 16-cyclopropyl-5α-androstan-17-one; 16-cyclobutyl-5-androsten-17-one; 16-hydroxymethylene-5-androsten-17-one; 3α-bromo-16α-methoxy-5-androsten-17-one; 16-oxymethylene-5-androsten-17-one; 3β-methyl-16ξ-trifluoromethyl-5α-androstan-17-one; 16-carbomethoxy-5-androsten-17-one; 3β-methyl-16β-methoxy-5α-androstan-17-one; 3β-hydroxy-16α-dimethylamino-5-androsten-17-one; 17α-methyl-5-androsten-17β-ol; 17α-ethynyl-5α-androstan-17β-ol; 17β-formyl-5α-androstan-17β-ol; 20,21-epoxy-5α-pregnan-17α-ol; 3β-hydroxy-20,21-epoxy-5α-pregnan-17α-ol; 16α-fluoro-17α-ethenyl-5-androsten-17β-ol; 16α-hydroxy-5-androsten-17α-ol; 16α-methyl-5α-androstan-17α-ol; 16α-methyl-16β-fluoro-5α-androstan-17α-ol; 16α-methyl-16β-fluoro-3-hydroxy-5-androsten-17-ol; 3β,16β-dimethyl-5-androsten-17β-ol; 3β,16,16-trimethyl-5-androsten-17β-ol; 3β,16,16-trimethyl-5-androsten-17-one; 3β-hydroxy-4α-methyl-5-androsten-17α-ol; 3β-hydroxy-4α-methyl-5-androsten-17-one; 3α-hydroxy-1α-methyl-5-androsten-17-one; 3α-ethoxy-5α-androstan-17β-ol; 5α-pregnan-20-one; 3β-methyl-5α-pregnan-20-one; 16α-methyl-5-pregnen-20-one; 16α-methyl-3β-hydroxy-5-pregnen-20-one; 17α-fluoro-5-pregnen-20-one; 21-fluoro-5α-pregnan-20-one; 17α-methyl-5-pregnen-20-one; 20-acetoxy-cis-17(20)-5α-pregnene; and 3α-methyl-16,17-epoxy-5-pregnen-20-one, for example.

In one aspect of the present invention, a DHEA congener and a second anti-inflammatory active agent can be co-administered to a subject in an amount that results in a therapeutic effect, thereby aiding in treating and/or preventing inflammation in a subject. The dose of the DHEA congener administered is selected to achieve DHEA or DHEA equivalent blood levels greater than normal endogenous DHEA blood levels. Normal endogenous blood levels of DHEA can be less than 20 ng/mL. Accordingly, peak blood levels of DHEA or DHEA equivalent can be greater than about 20 ng/mL, or as desired for a specific therapeutic effect. In another aspect, suitable doses that are selected to achieve a peak blood level of DHEA or DHEA equivalent can be in the range from about 30 ng/mL to about 100 mg/mL, or in the range from about 50 ng/mL to about 10 mg/mL. Additionally, the doses administered to a subject can be in an amount to achieve DHEA blood levels in the subject from about 100 ng/mL to about 1 mg/mL, from 100 ng/mL to about 100 μg/mL, and/or from about 100 ng/mL to about 10 μg/mL.

In accordance with the methods of the present invention, a DHEA congener can be administered as a part of a regimen to aid in the reduction of subchronic to chronic inflammation as well as acute inflammation. In one aspect, a DHEA congener can be administered in a dosing regimen that includes providing from about 10 mg to about 200 mg per day of the DHEA congener to a subject to aid in reducing subchronic to chronic inflammation. In another aspect, a DHEA congener can be administered in a dosing regimen that includes providing from about 10 mg to about 3600 mg of the DHEA congener to a subject to aid in reducing acute inflammation. In still a further aspect of the present invention, a DHEA congener can be administered in a dosing regimen to aid in preventing the onset of inflammation, which includes providing from about 10 mg to about 3600 mg per day of the DHEA congener to a subject not yet experiencing observable inflammation.

B) Anti-Inflammatory Active Agents for Administration with DHEA Congener for Treatment of Cancer

As described, DHEA congeners can be used in effective dosing regimens for reducing inflammation associated with cancer. As such, DHEA can be co-administered with any of a variety of anti-inflammatory active agents, referred to generally as “second anti-inflammatory agents,” in accordance with methods of the present invention to more effectively reduce inflammation or treat inflammatory producing diseases. Accordingly, the DHEA congener and the second anti-inflammatory agent can each be administered at a therapeutically effective dosage, such that the effects of cancer can be reduced or controlled more effectively than if each composition were administered alone.

A brief summary of several exemplary cancer and inflammation diseases associated with cancer are described herein, along with specific second anti-inflammatory agents that can be co-administered with DHEA to achieve a therapeutic effect. In accordance with these exemplary inflammatory conditions and exemplary effective anti-inflammatory agents for use in accordance with embodiments of the present invention, several specific dosage examples, dosage timing, dosage periods, administration routes, etc, are provided. However, it is to be understood that this information is provided for exemplary purposes only. These and other administration considerations can be varied by one skilled in the art (for administration with DHEA) in order to achieve a therapeutic effect. In other words, as one skilled in the art understands, dosages, timing of administration, length of administration, administration routes, drug selection, etc., should considered on a case-by-case basis. As an example of the variability that can exist, when considering the dosages provided herein with respect to the second anti-inflammatory agent, the dosages can be administered at from about 20% to about 500% of the dosages provided, for example. The lists of drugs presented herein are not considered to be exhaustive, and to the extent that a second anti-inflammatory agent can be administered with DHEA to enhance an anti-inflammatory response, that composition can be administered in accordance with embodiments of the present invention. When referring to dosages, typically, it is understood that the dosage amounts are for oral administration unless stated otherwise, or unless the context dictates otherwise.

i) Pancreatitis

Pancreatitis is a malady that can be treated by methods of the present invention. In an aspect of the present invention, methods of treating and/or preventing pancreatitis can include inhibiting pancreatic cancer growth. In another aspect, such methods that inhibit pancreatic cancer growth can slow the development of pancreatic cancer, and can slow pancreatic cancer cell proliferation. As such, methods of reducing inflammation in a subject, such as those including the co-administration of a DHEA congener and a second anti-inflammatory agent to the subject, can be used in treating and/or preventing pancreatitis. Such second anti-inflammatory active agents that can be delivered with a DHEA congener for the treatment and/or prevention of pancreatitis include COX-2 inhibitors, such as celecoxib, rofecoxib, valdecoxib, parecoxib, lumiracoxib, and combinations thereof.

ii) Pancreatic Cancer

As mentioned, in addition to treating pancreatitis, the methods of the present invention can result in inhibiting the growth of pancreatic cancer in a subject. Such methods can include co-administering a therapeutically effective amount of a DHEA congener and a COX-2 inhibitor to the subject. In an aspect of the present invention, the COX-2 inhibitors can include celecoxib, rofecoxib, valdecoxib, parecoxib, lumiracoxib, and combinations thereof. In another aspect, the methods of the present invention that inhibit the growth of pancreatic cancer can include preemptively co-administering the DHEA congener and the COX-2 inhibitor to the subject prior to being diagnosed with pancreatic cancer. In yet another aspect, preemptively co-administering the DHEA congener and the COX-2 inhibitor can reduce inflammation of the pancreas. In still another aspect, inhibiting the growth of pancreatic cancer can include treating a subject having pancreatic cancer. In still a further aspect of the present invention, inhibiting the growth of pancreatic cancer can include slowing pancreatic cancer cell proliferation.

C) Anti-TNF-α Agents for Administration with DHEA Congener

DHEA congeners can be used in effective dosing regimens for reducing inflammation, especially when co-administered with an anti-TNF-α agent. Therefore, anti-TNF-α agents may also serve as second anti-inflammatory agents for the purposes of the present invention. Such anti-TNF-α agents can include certain monoclonal antibodies, which may be chimeric anti-TNF-α(IgG1) monoclonal antibodies. For example, in one embodiment, such an anti-TNF-α agent that can be used includes infliximab. In accordance with embodiments of the present invention, the DHEA congener and the anti-TNF-α agent can each be administered to a subject at a therapeutically effective amount to preemptively inhibit inflammation, reduce inflammation, and/or otherwise treat the subject experiencing inflammation. In one aspect, the DHEA congener and the anti-TNF-α agent can each be administered at a therapeutically effective dosage in combination, such that the inflammation can be reduced more than each administered alone. Of course, any of the second anti-inflammatory agents described herein can additionally be co-administered at a therapeutically effective amount to the subject.

Without being bound to any particular theory, it is believed that the co-administration of the anti-TNF-α with DHEA can be effective by the inhibition of certain cell signaling pathways that are responsive to TNF-α. These cell signaling pathways can include NF-kappa-β, P38 MAP kinase, and combinations thereof. As such, when the NF-kappa-β and/or P38 MAP kinase cell signaling pathways are inhibited, there can be a decrease in TNF-α production and/or secretion. Thus, the systemic availability of TNF-α can become diminished, which may result in reduced inflammation. Furthermore, a decrease in available TNF-α may retard proliferation of cancer cells. While NF-kappa-β pathways can affect TNF-α production, there is also evidence that TNF-α can activate intracellular NF-kappa-β, completing a positive feedback loop. As such, there appears to be a connection between TNF-α levels and cancer, in that NF-kappa-β causes increased transcription of genes that contribute to tumor promotion and progression by affecting mechanisms governing programmed cell death.

Alternatively, there are indications that the up-regulation of TNF-α can increase the production and/or secretion of certain immune mediators that are responsive to TNF-α. These immune mediators can in turn stimulate an immunological response that increases inflammation. Accordingly, anti-TNF-α co-administered with DHEA can be effective in reducing inflammation by inhibiting the production and/or secretion of these immune mediators. These immune mediators that are responsive to TNF-α can include, without limitation, IL-1, IL-6, IL-3, G-CSF, GM-CSF, IL-10, IL-1Ra, IL-2, IL-4, IL-8, IL-12, IL-18, IFN-γ, and combinations thereof.

In accordance with methods of the present invention, the reduction of inflammation in a subject can be utilized to treat cancer and inflammatory diseases associated with cancer. To the extent that these diseases may be related to the production and/or secretion of TNF-α, the reduction of systemic TNF-α can reduce inflammation in the subject, and may retard the proliferation of cancer cells and tumor progression.

D) Preparation and Dosage Forms

Pharmaceutical compositions containing any compound of the present invention as the active ingredient can be prepared according to conventional pharmaceutical compounding techniques known to one of ordinary skill in the art. Typically, a therapeutically effective amount of the active ingredient can be admixed with a pharmaceutically acceptable carrier. The carrier may take a wide variety of forms depending on the desired route of administration, e.g., oral, intravenous, intrathecal epidural, transdermal, transbuccal, ocular, nasal, suppository. The compositions may further contain antioxidizing agents, stabilizing agents, preservatives, or the like.

Liquid carriers can be used in preparing solutions, suspensions, emulsions, syrups, elixirs and pressurized compositions. The active ingredients can be dissolved or suspended in a pharmaceutically acceptable liquid carrier such as water, cyclodextrins, an organic solvent, pharmaceutically acceptable oils, and/or fats. The liquid carrier can contain other suitable pharmaceutical additives such as solubilizers, emulsifiers, buffers, preservatives, sweeteners, flavoring agents, suspending agents, thickening agents, coloring agents, viscosity regulators, stabilizers and/or osmo-regulators. Suitable examples of liquid carriers for oral administration can include water, alcohols (including monohydric alcohols and polyhydric alcohols, e.g., glycols) and their derivatives, oils (e.g., peanut oil, sesame oil, olive oil, and coconut oil), and combinations of the above. Compositions comprising such carriers and adjuvants may be formulated using well-known conventional materials and methods.

A solid carrier can be formulated into capsules, pills, tablets, lozenges, melts, or powders. A solid carrier can include starches, sugars, bicarbonates, diluents, granulating agents, disintegrating, and/or dispersing agents. The formulations can include one or more substance(s) which may also act as flavoring agents, lubricants, solubilizers, suspending agents, fillers, glidants, compression aids, binders, or tablet-disintegrating agents, for example. In powders, the carrier can be a finely divided solid which is in an admixture with the finely divided active ingredients. The carrier and drug can form a single composite with drug adsorbed to its surface that effectively enhances the rate of dissolution in the gastrointestinal tract. The powders and/or tablet can contain up to 100 wt % of the active ingredients, though typically this will not be the case, and can be formulated for immediate and/or sustained release of the active ingredient.

With respect to tablets, the active ingredients can be mixed with a carrier having the necessary compression properties in suitable proportions and compacted in the shape and size desired. Exemplary forms can include dry powder compaction tablets, micro-particulate systems, e.g., wherein the active ingredient is spray-dried onto a scaffold particle, and hard or soft-gel capsules. In one embodiment, the tablets can be optionally covered with an enteric coating, which remains intact in the stomach, but will dissolve and release the contents of the tablet once it reaches the small intestine. Alternatively, the tablets can be formulated to enhance gastric uptake to avoid first pass effect in the liver following intestinal absorption.

The composition can include one or more sustained or controlled release excipient(s) such that a slow, sustained, or constant release of the active ingredients can be achieved. A wide variety of suitable excipients are known in the art. Such sustained/controlled release excipients and systems are described, for example, in U.S. Pat. Nos. 5,612,053; 5,554,387; 5,512,297; 5,478,574; and 5,472,711, each of which is incorporated by reference herein. If desired, the pharmaceutical composition can be formulated to provide a pulse dose of the active ingredient. A variety of pulse-dose systems, which provide low or high-pulsed doses, are known in the art. In another embodiment of the invention, the pharmaceutical can be formulated to provide direct and/or targeted delivery of the active ingredient to a specific anatomic site or sites within the gastrointestinal tract; e.g., the duodenum, jejunum, ileum, cecum and/or colon. Methods for providing targeted delivery of pharmaceuticals to specific tissues or organs within a mammalian host are well known in the art.

To achieve a therapeutic level in systemic circulation, a compound, i.e. active agent(s), can be formulated using standard techniques to form a composition having a high bioavailability of the active agents in order to meet the desired therapeutic blood levels. The active agent(s), a complex of the active agent(s) and cyclodextrin, or the active agent(s) in a nanoparticle delivery system may be dissolved in a pharmaceutical carrier and administered as either a solution or a suspension. Cyclodextrins of all classes (alpha, beta and gamma) and their substituted or derivatized forms can be used, as well as mixtures thereof. In one aspect of the present invention, a complex of the active agent(s) with a cyclodextrin or the active agent in a nanoparticle delivery system can be used. In another aspect, a complex of the active agent(s) and a cyclodextrin, such as a 2-hydroxypropyl β-cyclodextrin, can be prepared in accordance with U.S. Pat. No. 4,727,064 and/or European Patent No. 0 149 197, each incorporated herein by reference. The use of the compound as part of a cyclodextrin complex or nanoparticle delivery system can allow for the preparation of both parenteral and oral solutions and oral solid dosage forms with high concentration of active agent. Illustrative of suitable carriers include water, saline, dextrose solutions, fructose solutions, ethanol, or oils of animal, vegetative or synthetic origin. The carrier may also contain other ingredients including, for example, preservatives, suspending agents, solubilizing agents, buffers, and/or the like. When the compounds are being administered intrathecally, they or their cyclodextrin complexes or nanoparticle delivery systems may also be dissolved in cerebrospinal fluid.

The active agent can be administered in a therapeutically effective amount. The actual amount administered, and the rate and time-course of administration, can depend on the nature and severity of the condition being treated. Thus, it may be desirable to administer the highly bioavailable complex of the active agent at several intervals during a dosing regimen to maintain blood levels at the therapeutic index. For example, depending on the dosing regimen, it can be preferable to administer the formulated active agent two to four times per day, and more preferably to administer it two to three times per day. Alternatively, one might use an oral controlled release method to meter the drug available for absorption over a 24 hour period, thereby necessitating only a single dose per day. Prescription of treatment, e.g. decisions on dosage, timing, periods of administration, drug selection, etc., can be within the responsibility of general practitioners or specialists, and typically can take into account the disorder to be treated, the condition of the individual patient, the site of delivery, the method of administration, and other factors known to practitioners.

As will be appreciated by those of skill in the art, the form of the pharmaceutical composition of the active agent(s) and the mode of administration will determine the dose of the active agent to be delivered. A factor to consider in determining the proper dose to meet the desired peak blood levels is the bioavailability of the active agent in the pharmaceutical composition, i.e., the availability of the active agent for raising blood levels of DHEA or DHEA equivalent. For example, the bioavailability of the active agent(s) in a pharmaceutical composition delivered intravenously can be greater than that for the same pharmaceutical composition delivered orally. Thus, a lower dose of a pharmaceutical composition containing the active agent(s) can be administered intravenously than that which would be used orally. For oral administration of active agent(s) with low water solubility, it is well recognized that co-formulation of the agent(s) with a substance that accelerates dissolution or enhances solubility can increase the active agent's bioavailability. For example, active agent used to increase blood levels of DHEA congener or equivalent can be many times more soluble in water if complexed with a cyclodextrin than without. Similarly, the same active agent can dissolve in water much faster and have higher bioavailability if adsorbed to a high surface area particle with a large surface area. For example, suitable blood levels of the DHEA congener can be achieved by the administration of 100 mg of a cyclodextrin-DHEA complex intravenously, 600 mg of a cyclodextrin-DHEA complex orally, or 500 mg of DHEA in a nanoparticle delivery system orally. As will also be appreciated by those of skill in the art, the form of the pharmaceutical composition of the DHEA congener and the second active agent can depend on the intended mode of administration, which in turn will depend on the location and nature of the disorder to be treated. Accordingly, delivery to the gastrointestinal tract, e.g., for treatment of cancers associated with the gastrointestinal tract, can be in the form of oral solutions, gels, suspensions, tablets, capsules, and the like.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

### Inhibiting the Growth of Pancreatic Cancer Cells

The effects of DHEA and/or celecoxib on the proliferation of Capan-1 cells (HTB-79) in a pancreatic cell culture were studied. The Capan-1 cells were obtained from ATCC and grown in IMDM media supplemented with 20% FBS, 50 units/mL of penicillin, 50 mcg/mL of streptomycin prior to seeding. The cells were then harvested, counted, and seeded at a density of 2×10³ cells per well in a 96 well microtiter plate with 100 mcL of the media previously described. After 24 hours, the cells were observed to have adhered to the bottom of the wells. Accordingly, the media of each well was removed and replaced with 200 mcL of a test solution, where the different test solutions included various concentrations of DHEA alone, celecoxib alone, DHEA with celecoxib, and DMSO alone. The stock solutions were prepared by dissolving celecoxib and DHEA independently in different solutions of 100% DMSO. The stock solutions were then diluted into the growth media and adjusted to a total volume of 200 mcL of test solution.

The effects of the test solutions in each well after three days of incubation were determined using the CellTiter 96® AQueous One Solution Cell Proliferation Assay (Promega, Madison, Wis.). The procedure was conducted as follows: (1) CellTiter 96 AQueous ONe Solution Reagent was thawed at ambient temperature, (2) 20 mcL of the reagent was added to each well containing the Capan-1 cells and 100 mcL of test solution, (3) the 96 well plate was incubated for 4 hours at 37° C., 5% CO₂, and the absorbance at 490 nm was recording using a 96-well plate reader. As a note, the 200 mcL. The cell viability assay results for each test solution compared to a control were recorded, and are shown as set forth in Table 1, as follows:

TABLE 1
Cell viability in the presence of celecoxib and/or DHEA

| Celecoxib Dose (mcM) | DHEA Dose (mcM) | Cell viability (%) |
|---|---|---|
| 0 (mcM) | 0 (mcM) | 100% |
| 1 (mcM) | 0 (mcM) | 97% |
| 10 (mcM) | 0 (mcM) | 93% |
| 50 (mcM) | 0 (mcM) | 115% |
| 0 (mcM) | 1 (mcM) | 92% |
| 0 (mcM) | 10 (mcM) | 105% |
| 0 (mcM) | 50 (mcM) | 102% |
| 1 (mcM) | 1 (mcM) | 84% |
| 50 (mcM) | 1 (mcM) | 55% |
| 1 (mcM) | 50 (mcM) | 74% |
| 50 (mcM) | 50 (mcM) | 45% |

As can be seen by Table 1, the results indicate that no inhibition of Capan-1 cell growth was observed with the control (0 mcM Celecoxib and 0 mcM DHEA), and only minimal inhibition was observed with some specific doses of DHEA or celecoxib administered alone. However, the combination of DHEA and celecoxib resulted in significant inhibition of cell proliferation. The greatest inhibition of cell growth was observed with the test solution containing 50 mcM celecoxib and 50 mcM DHEA, which resulted in 55% inhibition of cell growth. The second greatest inhibition of cell growth was observed with 50 mcM celecoxib and 1 mcM DHEA, which resulted in 45% inhibition of cell growth.

### Example 2

### Effect of DHEA and/or Sulfasalazine on TNF-α In Vivo

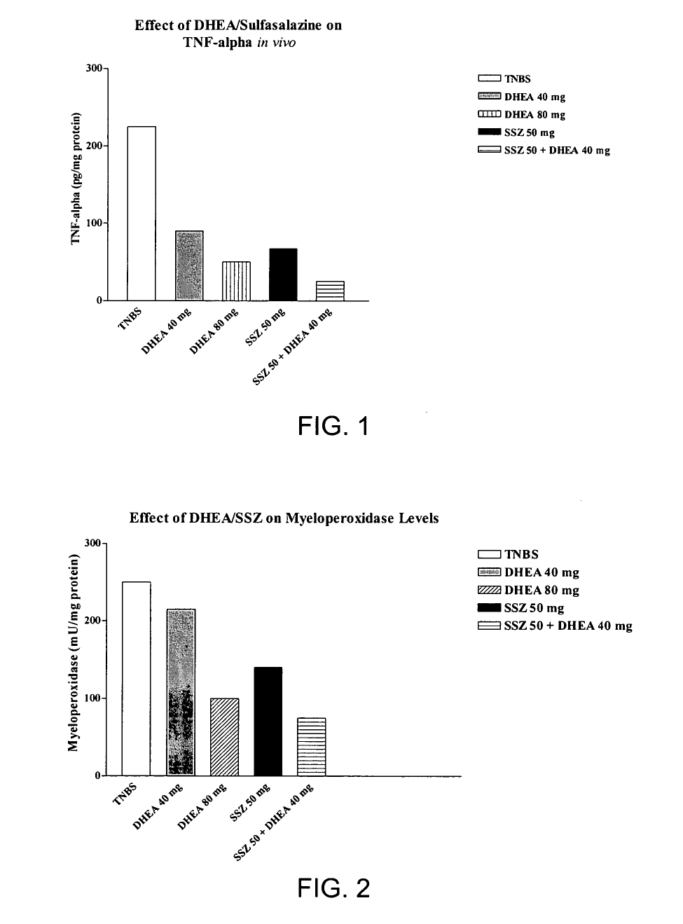

The effects of DHEA and/or sulfasalazine on a TNBS IBD animal model were studied by administering DHEA alone (40 mg/kg/day or 80 mg/kg/day), sulfasalazine (SSZ) alone (50 mg/kg/day), and a combination of DHEA and sulfasalazine (40 mg/kg/day DHEA with 50 mg/kg/day sulfasalazine). The intrarectal administration of TNBS (2,4,6-trinitrobenzene sulfonic acid) in mice/rats is a well-characterized animal model for human inflammatory bowel disease (IBD). The TNF-α levels (pg/mg protein, three days after challenge with intracolonic TNBS) for the treated animals were collected and compared to an untreated TNBS animal model. FIG. 1 illustrates the results. As can be seen from FIG. 1, a combination of DHEA with sulfasalazine works in a gold standard IBD animal model for reducing TNF-α levels better than either DHEA or sulfasalazine alone.

### Example 3

### Effect of DHEA and/or Sulfasalazine on Myeloperoxidase Levels

The effects of DHEA and/or sulfasalazine on a TNBS IBD animal model were studied by administering DHEA alone (40 mg/kg/day or 80 mg/kg/day), sulfasalazine (SSZ) alone (50 mg/kg/day), and a combination of DHEA and sulfasalazine (40 mg/kg/day DHEA with 50 mg/kg/day sulfasalazine). The intrarectal administration of TNBS (2,4,6-trinitrobenzene sulfonic acid) in mice/rats is a well-characterized animal model for human inflammatory bowel disease (IBD). The myeloperoxidase levels (mU/mg protein, three days after challenge with intracolonic TNBS) for the treated animals were collected and compared to an untreated TNBS animal model. Myeloperoxidase levels are a measure of white cell infiltrate to the site of inflammation. FIG. 2 illustrates the results. As can be seen from FIG. 2, a combination of DHEA with sulfasalazine works in a gold standard IBD animal model for reducing myeloperoxidase levels better than either DHEA or sulfasalazine alone.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A method of treating a subject with cancer or inflammation associated with cancer, comprising co-administering to the subject a therapeutically effective amount of a DHEA congener and a second anti-inflammatory agent.

2. A method as in claim 1, wherein the subject has been diagnosed with pancreatitis and is determined to be at risk of getting pancreatic cancer.

3. A method as in claim 1, wherein the subject has been diagnosed with pancreatic cancer.

4. A method as in claim 1, wherein the method of treating the subject includes slowing pancreatic cancer cell proliferation.

5. A method as in claim 1, wherein the method of treating the subject includes reversing the presence of pancreatic cancer.

6. A method as in claim 1, wherein the second anti-inflammatory agent is a COX-2 inhibitor.

7. A method as in claim 7, wherein the COX-2 inhibitor is selected from the group consisting of celecoxib, rofecoxib, valdecoxib, parecoxib, lumiracoxib, and combinations thereof.

8. A method as in claim 52, wherein the COX-2 inhibitor is celecoxib.

9. A method as in claim 8, wherein the COX-2 inhibitor is rofecoxib.

10. A method as in claim 1, wherein the DHEA congener and the second anti-inflammatory agent are each administered at a therapeutically effective dosage, such that the cancer cell proliferation is reduced more than by the summation of inflammation reduction for each administered alone.

11. A method as in claim 1, wherein the DHEA congener and the second anti-inflammatory agent are each administered at a therapeutically effective dosage, such that existing cancer cells are reduced in number more than by the summation of inflammation reduction for each administered alone.

12. A method as in claim 1, wherein the DHEA congener and the second anti-inflammatory agent are each administered at a therapeutically effective dosage, such that the cancer cell proliferation is reduced by more than if the same dosage of the DHEA congener or the second anti-inflammatory agent were administered alone.

13. A method as in claim 1, wherein the DHEA congener and the second anti-inflammatory agent are each administered at a therapeutically effective dosage, such that existing cancer cells are reduced in number more than if the same dosage of the DHEA congener or the second anti-inflammatory agent were administered alone.

14. A method as in claim 1, wherein the subject is human.

15. A method of inhibiting the growth of pancreatic cancer in a subject, comprising co-administering a therapeutically effective amount of a DHEA congener and a COX-2 inhibitor to the subject.

16. A method as in claim 15 wherein the COX-2 inhibitor is selected from the group consisting of celecoxib, rofecoxib, valdecoxib, parecoxib, lumiracoxib, and combinations thereof.

17. A method as in claim 15 wherein inhibiting the growth of pancreatic cancer includes preemptively co-administering the DHEA congener and the COX-2 inhibitor to the subject prior to being diagnosed with pancreatic cancer, wherein said subject has been determined to have a predisposition to pancreatic cancer.

18. A method as in claim 17 wherein preemptively co-administering the DHEA congener and the COX-2 inhibitor reduces inflammation of the pancreas.

19. A method as in claim 15, wherein inhibiting the growth of pancreatic cancer includes treating a subject having pancreatic cancer.

20. A method as in claim 15, wherein inhibiting the growth of pancreatic cancer includes slowing pancreatic cancer cell proliferation.

21. A method as in claim 15, wherein inhibiting the growth of pancreatic cancer includes reversing presence of pancreatic cancer cells.

22. A method as in claim 15, wherein the subject is human.
